(19) United States
(12) Patent Application Publication
(10) Pub. No.: US 2006/0025394 A1
(43) Pub. Date: Feb. 2, 2006

(54) Estradiol-related compounds and methods of use as anti-tumor agents

(76) Inventors: Shuk-Mei Ho, Lexington, MA (US); James A. Mobley, Nashville, TN (US)

Correspondence Address: FISH & RICHARDSON PC, P.O. BOX 1022, MINNEAPOLIS, MN, 55440-1022, US

(21) Appl. No.: 10/992,972

(22) Filed: Nov. 18, 2004

Related U.S. Application Data
(60) Provisional application No. 60/520,968, filed on Nov. 18, 2003.

(51) Int. Cl. A61K 31/56

(52) U.S. Cl. 514/182

(57) ABSTRACT

This invention relates to new estradiol-related compounds that can be used to treat various types of cancer including prostate and breast cancers.

### CROSS-REFERENCE TO RELATED APPLICATIONS

This application claims priority to U.S. Provisional Patent Application Ser. No. 60/520,968, filed on Nov. 18, 2003, the contents of which are hereby incorporated by reference in their entirety.

### STATEMENT AS TO FEDERALLY SPONSORED RESEARCH

This invention was made with Government support under NIH grant number DK61084, U.S. Army Prostate Research Program grant number DAMD17-98-1-8606, and NIH grant number CA81049. In addition, this invention was supported by Grant Number 5 P30 DK32520 from the National Institute of Diabetes and Digestive and Kidney Diseases. The government has certain rights in this application.

### TECHNICAL FIELD

This invention relates to new estradiol-related compounds that can be used to treat cancer.

### BACKGROUND

Prostate cancer (CaP) remains the number one cause of noncutaneous cancer, and the second leading cause of cancer-related deaths in American men with 220,900 new cases predicted for the year 2003 alone (American Cancer Society Inc., Cancer Facts and Figures 2003). For patients diagnosed with localized disease, treatments such as extirpative surgery and radiation therapy are associated with promising long-term outcomes. However, 15-20% of these patients will go on to present with advanced metastatic disease. The current treatment strategies for patients with metastatic CaP revolve around inhibition of androgen biosynthesis, and/or direct blockade of the androgen receptor. This approach, referred to as hormone therapy (HT), currently includes luteinizing hormone-releasing hormone (LHRH) agonists, androgen receptor antagonists, and direct inhibitors of androgen biosynthesis. While the classic HT approaches are known to lengthen the time to symptomatic onset, there is no significant increase in lifespan. This unfortunate observation is a result of the inevitable selection of cancer cells capable of proliferating independent of androgen stimulation. Treatment strategies following this stage are generally considered palliative, as no survival benefits have been documented in a significant patient population to date.

Therefore, a number of novel therapeutic strategies are actively being investigated. Of the more promising classes of compounds emerging for the treatment of CaP, estrogens of various types have exhibited anti-tumor activities both in vitro and in vivo. For example, estramustine, diethylstilbestrol (DES), raloxifene, genistein, resveratrol, and licochalcone are estrogens that have all shown to be promising agents for the treatment or prevention of CaP. Various such compounds are shown in FIG. 1. Interestingly, the mechanism(s) reported to be responsible for the noted anti-tumor properties of this class are numerous, and yet have not been thoroughly defined in vivo.

Most estrogens act primarily on the hypothalamic-pituitary axis, thereby inhibiting activation of testosterone synthesis. However, many estrogens including DES, 2-methoxy-E₂, and estramustine have been found to exhibit a level of anti-tumor activity that is likely to be independent ofthis pathway. In addition, the pure anti-estrogen compound ICI-182,780 and the selective estrogen receptor modulator Raloxifene have been reported to decrease cell number and induce apoptosis in CaP cell lines in vitro through what has been described as an estrogen receptor beta (ER-β) mediated mechanism. Unfortunately, no completely effective treatments have yet been discovered.

### SUMMARY

The invention is based, at least in part, on the discovery that a certain class of estradiol-related compounds is effective as anti-tumor agents at very low dosages. In particular, 17α-20Z-21-[(4-amino)phenyl]-19-norpregna-1,3,5(10),20-tetraene-3,17β-diol (referred to herein as APVE₂), is a useful compound within this class. These compounds induce high levels of cell death in various prostate and breast cancer cell lines, and can be used to treat excess cell proliferation caused by various types of tumors throughout the body.

In general, the invention features 2,3,4-substituted-E/Z-phenylvinyl-17β-estradiol analogs of the structure:

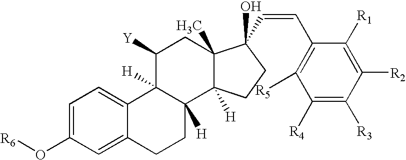

In this formula, R₆ can be H, any alkyl, or hetero-containing alkyl. R₁, R₂, R₃, R₄, and R₅ can be, independently, H, halo (e.g., F, Cl, Br), alkyl, hetero-containing alkyl, NH₂, OH, SH, NHR₆, NR₆, OR₆, NO₂, or SR₆. In some embodiments, R₆ can be H, or CHR₇R₈, where each of R₇ and R₈ is a hydrogen, any heteroatom (e.g., amino, oxygen, sulfur), a linear or cyclic, conjugated or saturated, homo or hetero, or an alkyl (CHₓ)ₙ group, where x=1 to 20, and n is any number. Y is an alkoxy group containing up to about 15 carbon atoms, e.g., methoxy, ethoxy, n-propoxy, t-butoxy, neopentoxy or n-hexoxy. Different compounds covered by this structure include 4-substituted-Z-phenylvinyl-17β estradiol (4-substituted-Z-PVE₂), p-amino-Z-PVE₂, and APVE₂.

The invention also features new methods of inhibiting, e.g., decreasing, cell proliferation (e.g., methods of treating cancer) in a subject, such as a mammal, e.g., a human, or a domesticated mammal such as a dog, cat, mouse, rat, primate, horse, cow, sheep, or pig; by identifying a subject in which a decrease (or prevention or reduction of an increase) in cell proliferation is desirable; and administering to the subject one of the new compounds described herein in an effective amount.

In various embodiments of the formula above, R₆ can be hydrogen; R₁, R₂, R₄, and R₅ can be H and R₃ can NH₂. In various compounds, the substituents can be as follows: R₁, R₃, R₄, and R₅ are H, R₂ is CH₃; R₁, R₂, R₄, and R₅ are H, R₃ is F; R₂, R₃, R₄, and R₅ are H, R₁ is CF₃; R₁, R₃, R₄, and R₅ are H, R₂ is CF₃; or R₁, R₂, R₄, and R₅ are H, R₃ is CF₃.

The compound can be administered intramuscularly, systemically, via an implant, e.g., one that provides sustained release of the compound, or the compound can be administered intravenously. The cell proliferation can be associated with cancer, e.g., prostate or breast cancer. In certain embodiments, the amount of the compound administered is about 0.1 to 20 μg/kg body weight of the mammal per day. In other embodiments, the compound is administered in the range of from about 0.1 μg to about 40 mg/kg/day.

In particular embodiments, the compound is 17α-20Z-21-[(4-amino)phenyl]-19-norpregna-1,3,5(10),20-tetraene-3,17β-diol.

In a more specific embodiment, the invention also features methods of treating cancer, the method comprising administering to an individual having a cancer or at risk for having a cancer an amount of 17α-20Z-21-[(4-amino)phenyl]-19-norpregna-1,3,5(10),20-tetraene-3,17β-diol effective to inhibit proliferation of cells of the cancer.

In different embodiments, the new compounds can be 4-substitued-Z-phenylvinyl-17β estradiol, p-amino-Z-phenylvinyl-17β estradiol, or 17α-20Z-21-[(4-amino)phenyl]-19-norpregna-1,3,5(10),20-tetraene-3,17β-diol. The invention also includes new compositions that include the new compounds and a pharmaceutically acceptable carrier, such as sterile saline, purified water, fixed oils, polyethylene glycols, glycerin, propylene glycol, poly(lactide-co-glycolide), polyacrylate, latex, starch, cellulose, or dextran.

As used herein, an effective amount of a compound provides a measurable improvement in a subject's condition, i.e., by reducing the size of a solid tumor at least 20% compared to a size measured prior to administration of the compound or by reducing the amount of cell proliferation in a growing tumor by at least 20% compared to a level measured prior to administration. The level of cell proliferation of a tumor and the size of a tumor can be measured using standard techniques. An effective amount of a compound can also be an amount that maintains a normal level of cell proliferation in a tissue that has a risk or potential, if untreated, to become cancerous.

As used herein, “inhibiting cell proliferation” or “inhibiting proliferation of cells” includes decreasing cell proliferation, e.g., in an active or growing tumor, and reducing or preventing an undesirable increase in cell proliferation, e.g., in a tissue that may, absent treatment, undergo an increase in cell proliferation and become cancerous.

The invention has several advantages. For example, the low-nanomolar concentrations required for activity in cell culture models, in conjunction with reasonable chemical stability, indicate that reaching pharmacological doses of the new compounds should not require special drug delivery applications. In addition, the chemistry used for the synthesis of the new compounds allow for ease of scale-up. General toxicity will likely be low, based on tests of similar drugs in this class, which do not induce any apparent general or organ-specific damage at doses far greater than that required for maximal anti-neoplastic activity of the new compounds.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a representation of various synthetic and natural estrogens that exhibit anti-tumor activities. These structures mimic E₂ (estradiol-17β) with a rigid planar hydroxylated biaryl geometry similar to that of the 3- and 17β-positions on the A and D rings of E₂.

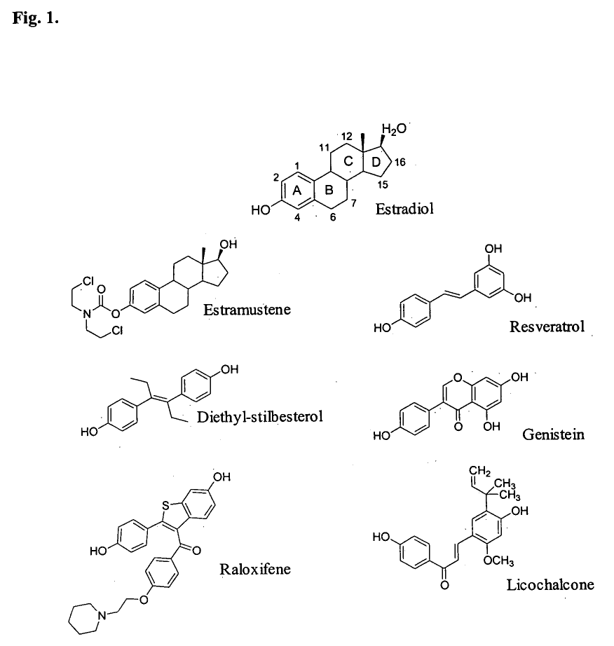

FIG. 2 is a representation of an energy-minimized depiction of APVE₂. The compound is shown bound within the ligand-binding pocket (LBP) of ER-β (light gray) and ER-α (dark gray). Hydrogen bonds are illustrated as dotted lines. The phenyl moiety was predicted to cause a higher level of steric hindrance within the LBP of ER-α as a result of Met 421 in the LBP, as opposed to the corresponding Ile 373 in the LBP for ER-β.

FIG. 3 is a representation of a synthesis scheme for 17α-Z-4-aminophenylvinyl-17β-estradiol (APVE₂) (5).

FIG. 4 is a graph showing the change in cell number after a five day treatment with varying concentrations of APVE₂ as compared to the carrier treated control for three commonly utilized CaP cell lines (mean, standard deviation, n=4). The effective concentrations at 50% of the maximum cell death (EC₅₀'s) are illustrated above for APVE₂ as well as other pharmacologically active estrogens based on values derived from literature (Dahllof et al, Cancer Res., 53:4573-4581, 1993; Robertson et al., J. Natl. Cancer Inst., 88:908-917, 1996; Kumar et al., J. Mol. Carcinog., 31:111-124, 2001; Kyle et al., Mol. Pharmacol., 51:193-200, 1997).

FIG. 5 is a bar graph showing the change in cell number after a five day treatment with 1.0 μM and the EC₅₀ concentration of 16 nM APVE₂ alone, 1.0 μM E₂ alone, and both as compared to the carrier treated control in DU145 cells (n=4, *p<0.05). The cytotoxic action of 16 nM APVE₂ was not effected by co-treating with 1 μM E₂ at that same time or pre-treating the cells 1 hour prior to the addition of APVE₂ as shown.

FIGS. 6A and 6B are a pair of blots of recombinant ER-αᵣ and ER-βᵣ used to test the specificity of each antibody. The resultant blots indicate high specific binding and the resultant bands correlate with the expected molecular weights as shown at 54 (ER-α) and 69 kDa (ER-β).

FIGS. 7A and 7B are a pair of gels. Each lane was loaded with 25 μg of protein extract from DU145 cells. High levels of ER-β and barely measurable levels of ER-α were found to be expressed under the defined media conditions as shown above (n=3). β-Actin was used to control for equal loading and 10 μg of protein extract from MCF-7 cells were used as a positive control for ER-α.

FIGS. 8A and 8B are graphs showing cell cycle analysis including an annexin V assay on DU-145 cells after treatment with 16 nM APVE₂ over various time points (n=3). FIG. 8A shows accumulation in the G1 phase of 135% by the second and third day of treatment with a concomitant loss in S phase by up to 68%. Treatment for four to five days led to an increase in G2/M phase by 420% with a loss of G1 by over 48%. FIG. 8B shows the results of treatments with 16 nM APVE2, which caused a significant increase in late stage apoptosis of up to 79% by the day four, which correlated with the switch to G2/M and a change in cellular morphology.

### DETAILED DESCRIPTION

The invention is based, at least in part, on the discovery that a certain class of estradiol-related compounds, 2,3,4-substituted-E/Z-phenylvinyl-17β-estradiol analogs, is effective as anti-tumor agents at very low dosages. In particular, one compound, 17α-20Z-21-[(4-amino)phenyl]-19-norpregna-1,3,5(10),20-tetraene-3,17β-diol (referred to herein as APVE₂), is a useful compound in this class. This compound induces a high level (>90%) of cell death through an apoptotic mechanism, with an EC₅₀ of 1.4, 2.7, and 16 nM in the LNCaP, PC3, and DU145 cell lines, respectively. Additional studies indicated that this compound exhibited equal efficacies toward a number of breast cancer derived cells lines as well. This level of cancer-specific cytotoxic activity to multiple cancer cell lines is highly unusual, and therefore makes APVE₂ a very important novel addition to the currently known anti-tumor treatments for various solid malignancies in use today. In addition, the fact that the new compounds appear not to utilize an estrogen receptor-based mechanism makes these compounds even more versatile for use against solid malignancies that do not express this receptor at appreciable levels. Moreover, estrogens may exert a great variety of side effects such as gynaecomastia in men, cervical abnormality in women, promote breast cancer development, undesirable lipid profile, venus thromboembolism, and vaginal bleeding.

The new 2,3,4-substituted-E/Z-phenylvinyl-17β-estradiol analogs, including APVE₂, are novel and useful therapeutic agents for the following reasons: 1) these compounds induce apoptotic activities at low nanomolar concentrations; 2) they are effective against multiple cancer derived cell lines; 3) they exhibit a dose-related toxicity curve; 4) the cytotoxic actions of APVE₂ involve a non-estrogen receptor related pathway; and 5) due to the ionic properties of the para-substitution (e.g., para-amino substitution) on the phenyl group, these compounds exhibit a partition coefficient that allows delivery via intramuscular injection, as opposed to more complicated delivery systems such as microlipid encapsulation often necessary for highly lipophilic drugs.

In addition, some of the new compounds, such as APVE₂, have a Z conformation, which has not previously been shown to have a high level of activity.

New Compounds

The new compounds have the following general structure:

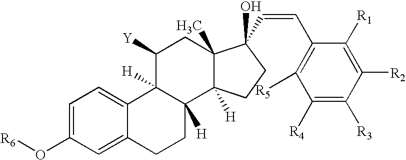

In this formula, R₆ can be H, any alkyl, or hetero-containing alkyl. R₁, R₂, R₃, R₄, and R₅ can be, independently, H, halo (e.g., F, Cl, Br), alkyl, hetero-containing alkyl, NH₂, OH, SH, NHR₆, NR₆, OR₆, NO₂, or SR₆. In some embodiments, R₆ can be H, or CHR₇R₈, where each of R₇ and R₈ is a hydrogen, any heteroatom (e.g., amino, oxygen, sulfur), a linear or cyclic, conjugated or saturated, homo or hetero, or an alkyl (CHₓ)ₙ group, where x=1 to 20, and n is any number. Y is an alkoxy group containing up to about 15 carbon atoms, e.g., methoxy, ethoxy, n-propoxy, t-butoxy, neopentoxy or n-hexoxy.

One of the new compounds, APVE₂, has the following structure:

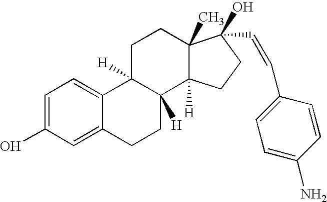

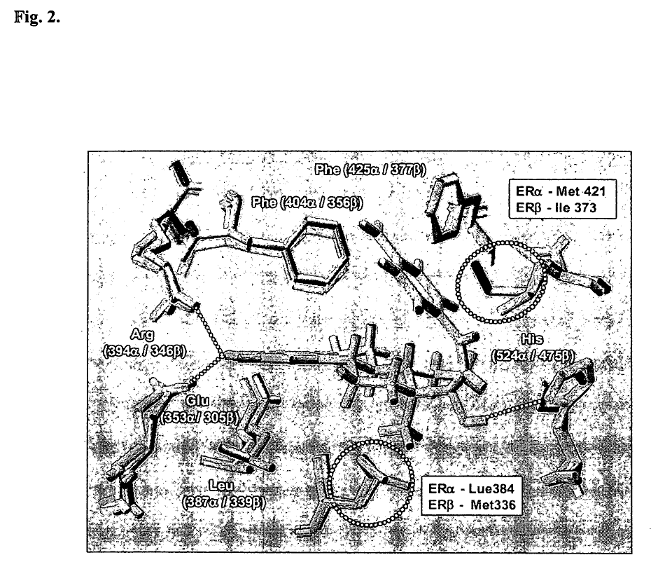

FIG. 2 is a representation of an energy-minimized depiction of APVE₂. The compound is shown bound within the ligand-binding pocket (LBP) of ER-β (light gray) and ER-α (dark gray). Hydrogen bonds are illustrated as dotted lines. The phenyl moiety was predicted to cause a higher level of steric hindrance within the LBP of ER-α as a result of Met 421 in the LBP as opposed to the corresponding Ile 373 in the LBP for ER-β.

The new compounds can be made using general synthetic methods as described in PCT WO 01/98322, which is incorporated herein by reference in its entirety. However, certain of the new compounds require specific alterations to the general synthetic methods described in PCT WO 01/98322, as described herein.

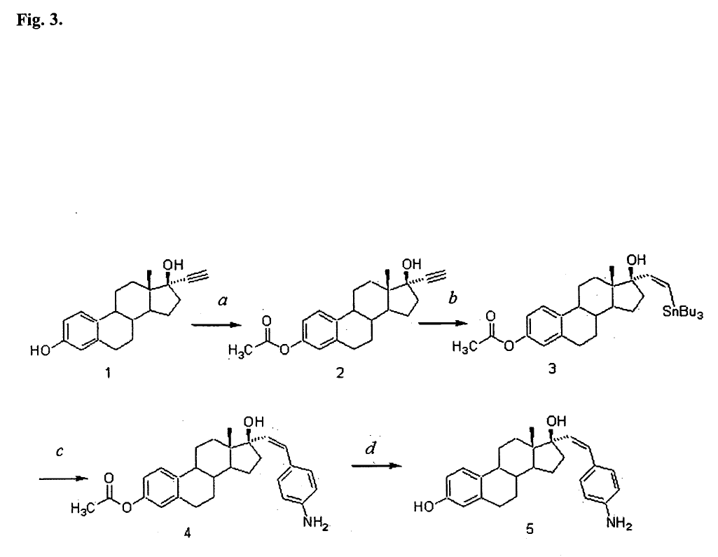

One useful synthetic method is illustrated in FIG. 3 and is described in detail in Example 1, below. In general, the following steps are illustrated in FIG. 3:
a) 17α-ethynylestradiol (EE) (1) is stirred with excess acetic anhydride in pyridine. The reaction mixture is poured into ice water, stirred and filtered.
b) The filtrate is collected and recrystallized from a solvent such as hexane:acetone to give the carboxylate ester (2). The cis-vinyl-tributyltin-EE isomer is then prepared by adding tri-n-butyl tin hydride and 1M triethylborane to a solution containing (2) in a solvent such as tetrahydrofuran (THF). The reaction is stirred without oxygen, e.g., under nitrogen. The THF is removed and the resulting oil is separated e.g., via silica gel column chromatography using hexane:ethyl acetate as both the packing and eluting solvent.
c) A mixture of p-iodoaniline is stirred with Tetrakis (triphenylphosphine) palladium, in refluxing anhydrous toluene. A solution of (3) and crystal of 2,6-di-tert-butyl-4-methylphenol in toluene is added. The reaction mixture is refluxed under nitrogen and cooled. A 10% KF/H₂O solution is added and the mixture is stirred. The solution is filtered to remove Pd black then the solution is partitioned between ethyl acetate:water. The aqueous layer is extracted with ethyl acetate. Organic layers can be combined and washed with brine and water, dried over magnesium sulfate, and concentrated. The residue is chromatographed on silica gel eluted with hexane:ethyl acetate to afford (4).
d) The crude acetylated product is stirred in a solution containing 10 N sodium hydroxide in methanol and acidified with 4% acetic acid. The deprotected product is then partitioned between ethyl acetate and water, dried over magnesium sulfate, and purified by column chromatography (5), followed by three rounds of recrystallization from acetone:chloroform.

Pharmaceutical Compositions and Methods of Administration

In some embodiments, the new compounds disclosed herein are mixed with pharmaceutically-acceptable carriers to form a pharmaceutical composition for administration to a cell or an animal, either alone, or in combination with one or more other modalities of therapy.

A pharmaceutical composition is formulated to be compatible with its intended route of administration. Examples of routes of administration include parenteral (e.g., intravenous, intramuscular, intradermal, subcutaneous), oral (e.g., inhalation), intranasal, transdermal (e.g., topical), transmucosal, and rectal administration. Solutions or suspensions used for parenteral, intradermal, or subcutaneous application can include the following components: a sterile diluent for injection such as water,- saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Carriers and Buffers

It will be understood that, if desired, a composition as disclosed herein may be administered in combination with other agents as well, such as, e.g., other anti-tumor agents. The compositions may thus be delivered along with various other agents as required in the particular instance. Such compositions may be purified after chemical synthesis.

It will be apparent that any of the pharmaceutical compositions described herein can contain pharmaceutically acceptable salts of the new compounds. Such salts can be prepared, for example, from pharmaceutically acceptable non-toxic bases, including organic bases (e.g., salts of primary, secondary, and tertiary amines and basic amino acids) and inorganic bases (e.g., sodium, potassium, lithium, ammonium, calcium, and magnesium salts).

Carriers for use within such pharmaceutical compositions are biocompatible, and may also be biodegradable. In certain embodiments, the formulation may provide a relatively constant level of active compound release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran, and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701, and WO 96/06638). The amount of active compound contained within a sustained-release formulation depends upon the site of implantation, the rate and expected duration of release, and the nature of the condition to be treated or prevented.

The new pharmaceutical compositions will often further comprise one or more buffers (e.g., neutral buffered saline or phosphate buffered saline), carbohydrates (e.g., glucose, mannose, sucrose, or dextrans), mannitol, proteins, gangliosides, or amino acids such as glycine, antioxidants, bacteriostats, chelating agents such as EDTA or glutathione, adjuvants (e.g., aluminum hydroxide), solutes that render the formulation isotonic, hypotonic, or weakly hypertonic with the blood of a recipient, suspending agents, thickening agents, and/or preservatives. Alternatively, compositions of the present invention may be formulated as a lyophilizate.

Packaging

The pharmaceutical compositions described herein may be presented in unit-dose or multi-dose containers, such as sealed ampoules or vials, along with instructions for use, e.g., to treat a specific cancer. Such containers are typically sealed in such a way to preserve the sterility and stability of the formulation until use. In general, formulations may be stored as suspensions, solutions, or emulsions in oily or aqueous vehicles. Alternatively, a pharmaceutical composition may be stored in a freeze-dried condition requiring only the addition of a sterile liquid carrier immediately prior to use.

Dosing, Delivery, and Treatment Regimens

The development of suitable dosing and treatment regimens for using the particular compositions described herein in a variety of treatment regimens, including e.g., oral, parenteral, intravenous, intranasal, and intramuscular administration and formulation, is well known in the art; some of these regimens are briefly discussed below for general purposes of illustration.

In certain circumstances, it will be desirable to deliver the pharmaceutical compositions disclosed herein systemically (e.g., parenterally, intravenously), intramuscularly, or intraperitoneally. Such approaches are well known to the skilled artisan. In certain embodiments, solutions of the active compounds as free bases or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases, the form must be sterile and must be fluid to the extent that easy syringability exists. It must be stable under the conditions of manufacture and storage and must be preserved against the action of contaminating microorganisms, such as bacteria and fungi. The carrier can be a solvent or dispersion medium containing, for example, water, ethanol, polyol (e.g., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation and in such amount as is therapeutically effective.

The phrase “pharmaceutically-acceptable” refers to molecular entities and compositions that do not produce an allergic or other untoward reaction when administered to a human.

The compositions described herein may be used in therapeutic methods for the treatment of cancer, e.g., in mammalian, e.g., human, patients. Such pharmaceutical compositions may be administered either prior to or following surgical removal of primary tumors and/or treatment such as administration of radiotherapy or conventional chemotherapeutic drugs. Routes and frequency of administration of the therapeutic compositions of the present invention, as well as dosage, will vary from individual to individual, and may be readily established using standard techniques. In general, the pharmaceutical compositions may be administered by injection (e.g., intracutaneous, intramuscular, intravenous, or subcutaneous), intranasally (e.g., by aspiration), or orally.

An appropriate dosage and treatment regimen provides the active compound(s) in an amount sufficient to provide therapeutic and/or prophylactic benefit. Such a response can be monitored by establishing an improved clinical outcome (e.g., more frequent remissions, complete, partial, or longer disease-free survival) in treated patients as compared to non-treated patients. Typically, a suitable dose is an amount of a compound that, when administered as described above, is capable of promoting an anti-tumor response, and is at least 10-50% above the basal (i.e., untreated) level.

The most effective mode of administration and dosage regimen for the compositions of this invention depends upon the severity and course of the disease, the patient's health and response to treatment, and the judgment of the treating physician. Accordingly, the dosages of the compositions should be titrated to the individual patient. An effective dose of the new compounds is in the range of from about 0.1 μg to about 40 mg per kilogram per day, e.g., 10 μg to 10 mg/kg/day, or 100 μg to 1.0 mg/kg/day. The new compounds should also work at about 0.0005 to 0.01, e.g., 0.001, mg/kg/day. Local administration can be about 1.0 to about 500 μg, or about 10 to 200 μg.

Before administration into humans, the new compounds and pharmaceutical compositions can be tested for biological activity (i.e., ability to decrease cell proliferation) both in vitro and in vivo. In vitro testing can be performed as described herein. In vivo animal models for tumor growth are well known, such as described in Nagane et al., Cancer Res., 60:847-53, 2000 and Price et al., Clin. Cancer Res., 5:845-54, 1999. Other models are described in Wechter et al., Cancer Res., 60(8):2203-8, 2000; Gleave et al., Cancer Res., 52(6):1598-605, 1992; Thalmann et al., Prostate, 44(2):91-103, 2000; Wu et al., Int. J. Cancer, 77(6):887-94, 1998.

Kits

The invention also encompasses kits for carrying out the new methods. The kits include (a) a pharmaceutical composition or compound described herein, and (b) instructions for use, e.g., specifying particular dosages and regimens. For example, the new compounds can be administered once or twice a day, or once every 3, 4, 5, 6, or 7 days, or even monthly, for up to two, three, four, or more months. The new compounds are orally active. The kit may also include ancillary agents such as buffering agents and stabilizing agents.

Uses of the New Compounds

The outcome of the examples below indicate that APVE₂ is an exceptionally potent anti-tumor agent, found to induce cell death through an apoptotic mechanism with EC₅₀ values of 1.4, 2.7, and 16 nM in the LNCaP, PC3, and DU145 cell lines, respectively. This is an important finding, since most pro-apoptotic estrogens including DES are active in the μM range, three orders of magnitude greater than that exhibited by APVE₂ (Rafi et al., Anticancer Res., 20: 2653-2658, 2000; Tinley et al., Cancer Res., 63: 1538-1549, 2003; Dahllofet al., Cancer Res., 53:4573-4581, 1993; Brueggemeier et al., J. Steroid Biochem. Mol. Biol., 78: 145-156, 2001; Robertson et al., J. Natl. Cancer Inst., 88: 908-917, 1996). In addition, this compound was capable of inducing apoptosis independent of p53, which has been shown to be mutated in the DU145 and PC3 cell lines (Webber et al., Prostate, 30: 58-64, 1997). All of the cell cycle studies including the apoptosis assay were carried out at the EC₅₀ concentration in DU145 cells of 16 nM to avoid induction of general cytotoxic effects or any other unforeseen cross reactive pathways. The cell cycle analysis studies revealed an increase in G1 arrest by days 2 and 3, which was followed by a pronounced increase (over 4-fold) in G2/M accumulation and a 79% increase in apoptosis and decreased adhesion. APVE₂ was found to bind weakly to ER-β with an EC₅₀ of 250 nM and a relative binding activity of 6.2%, a concentration far less than that required for cytotoxic activity. Furthermore, the cytotoxic actions of APVE₂ were not reversed by co-treating cells with a 50-fold excess concentration of E₂.

The combination of weak affinity for ER-β, cytotoxic effects in the low nanomolar range, and the inability to compete-out activity with E₂ is not consistent with an ER-β-dependent mechanism in mediating apoptotic induction by this compound. In addition, as predicted in silico, there was a preferential affinity for ER-β by 7-fold over that for ER-α, likely due to steric restrictions that correspond to amino acid Met 421 residing within the LBP of ER-α. This data also indicates that this class of E₂ analogs may prove to be useful probes for the study of ER selective ligands. Furthermore, there was no growth response exhibited by APVE₂ in the MCF-7 cell line (results not shown), indicating that this compound is not a classic ER-α agonist.

These results indicate that APVE₂ does not induce apoptosis through an ER-β mediated mechanism. Nevertheless, the results are striking, and the lack of ER-α activity potentially avoids the common undesirable side effects of estrogen agonists such as increased thrombosis, cardiotoxicity, and decreased libido (Carcinoma of the Prostate. In P. Walsh et al., (eds.), Campbell Urology, 8th ed W. B. Saunders, 2002; el Rayes, B. F. and Hussain, M. H. Expert. Rev. Anticancer Ther., 2:37-47, 2002. Results from flow cytometry analysis were consistent with mitotic spindle disruption.

To date, only one estrogen-related compound reported thus far, raloxifene, has exhibited activity nearing low nanomolar concentrations (Kim et al., J. Cancer Res., 62: 5365-5369, 2002). Therefore, the ability to achieve cell killing at 1.4 to 16 nM in CaP cells that exhibit varied levels of resistance to apoptosis, including the P53-mutated DU145 and PC3 cell lines, is of great interest (Martel et al., Cancer Treat. Rev., 29:171-187, 2003).

### EXAMPLES

The invention is further described in the following examples, which do not limit the scope of the invention described in the claims. In these examples, all statistical calculations, EC50 values, mean ±SD, and P values were carried out on GraphPad® Prism® software version 2.0, and calculated using a non-linear regression fit and the unpaired t-test at 95% confidence interval respectively.

### Example 1—General Methods of Synthesis

All reagents and solvents were purchased Sigma-Aldrich (Saint Louis, Mo.) or Fisher Scientific. The synthesis scheme is outlined in FIG. 3.

### 3-Acetoxy-(17α-20Z)-21-(tri-n-butylstannyl)19-norpregna-1,3,5(10)20-tetraene-17β-ol (3)

Protection of the 3-OH phenol was carried out as previously described with changes (Hanson et al., J. Med. Chem., 46:2865-2876, 2003; Counsell et al., J. Med. Chem., 9:689-692, 1966). Briefly, 17α-ethynylestradiol (EE) (1) (1.7 g, 5.7 mmol) was stirred with excess acetic anhydride (1 ml) in pyridine (15 ml) at room temperature (RT) for 12 hours. The reaction mixture was poured into ice water, stirred for 2 hours, and filtered. The filtrate was collected and recrystallized from hexane:acetone to give the carboxylate ester (2) as glassy crystals (1.8 g, 93%). The cis-vinyl-tributyltin-EE isomer was prepared for the following coupling step as previously reported with modifications. Briefly, to a solution containing (2) (6.76 g, 20 mmol) in tetrahydrofuran (THF) (20 ml), was added tri-n-butyl tin hydride (8.5 ml, 31 mmol) and 1M triethylborane (1 ml, 8.8 mmol). The reaction was stirred under nitrogen at room temperature for 10 hours. The THF was removed under reduced pressure and the resulting oil was separated via silica gel column chromatography using hexane:ethyl acetate (5:1) as both the packing and eluting solvent. Product (3) was isolated as an oil, solidifying upon standing (4.6 g, 36%; 63% based on recovered starting material; R_{f}=0.68 (hexane:ethyl acetate 5:1); mp 80-82° C.).

### 17α, 20Z-21-[(4-amino)phenyl]-19-norpregna-1,3,5(10),20-tetraene-3,17β-diol (APVE₂) (5)

The Stille coupling and deprotection were carried out as previously described with modifications (Hanson et al., supra). A mixture of p-iodoaniline (0.22 g, 5.7 mmol) was stirred with Tetrakis (triphenylphosphine) palladium (0.028 g, 0.02 mmol), in refluxing anhydrous toluene (25 ml) for 20 minutes. A solution of (3) (1.7 g, 2.8 mmol) and crystal of 2,6-di-tert-butyl-4-methylphenol in toluene (20 ml) was added. The reaction mixture was refluxed under nitrogen for 17 hours and cooled to room temperature. A 10% KF/H₂O (20 ml) solution was added and the mixture was stirred for one hour. The solution was filtered to remove Pd black, no SnBu₃F precipitated, then the solution was partitioned between ethyl acetate:water (100:100 ml).

The aqueous layer was extracted with ethyl acetate (2×50 ml). Organic layers were combined and washed with brine (100 ml) and water (2×100 ml), dried over magnesium sulfate, and concentrated. The residue was chromatographed on silica gel eluted with hexane:ethyl acetate (4:1) to afford (4) (0.06 g pure and 0.5 g mixture >35%): R_{f}=0.32 (hexane:ethyl acetate 5:1).

The crude acetylated product (˜5 g) was stirred for two hours in a solution containing 10 N sodium hydroxide in methanol and acidified with 4% acetic acid. The deprotected product was then partitioned between ethyl acetate and water, dried over magnesium sulfate, and purified by column chromatography to yield a dark orange solid (5) (0.34 g, 75.3%) followed by three rounds of recrystallization from acetone:chloroform to yield a light orange powder; (0.31 g, 68%).

The following test results were obtained: R_{f}=0.47 (1:1 hexane:ethyl acetate); mp 140-142° C.; ¹H NMR (CDCl₃): 0.91 (s, 3H, 18-CH₃), 1.2-2.9 (m, 15H, steroid nucleus), 3.8 (s, b, 2H, —NH₂), 4.6 (s, b, 17-OH), 5.79 (d, 1H, J₂₀₋₂₁=12.90 Hz, 20-H), 6.40 (d, 1H, J₂₁₋₂₀=12.60 Hz, 20-H), 6.56 (d, 1H, J₄₋₂=2.7 Hz, 4-H), 6.64 (m, 4H, 2-H, 4-H, 24-H and 26-H), 7.16 (d, 1H, J₁₋₂=8.4 Hz, 1-H), 7.26 (s, CDCl₃), 7.32 (d, 2H, J=8.4 Hz, 23-H and 27-H); ¹³C NMR (acetone d₆) 15.04 (C-18), 24.17 (C-15), 27.79 (C-11), 28.66 (C-7), under acetone peak (C-6), 32.94 (C-12), 38.51 (C-16), 41.25 (C-8), 45.03 (C-9), 49.12 (C-13), 50.12 (C-14), 83.99 (C-17), 113.90 (C-2), 114.58 (C-24 and C-26), 116.25 (C-4), 127.41 (C-1), 131.14 (C-21), 132.33 (C-10), 132.66 (C-23 and C-27), 133.03 (C-20), 138.76 (C-5), 150.02 (C-25), 156.29 (C-3).

The purified compound (5) has a mass of 389.24, a MW of 389.53, and has the formula C₂₆H₃₁NO₂. CLogP=4.919 and CMR=11.7203.

### Molecular Modeling

Local energy minima conformers of APVE₂ were initially determined using Chemdraw 3D v.7.0 (CambridgeSoft, Cambridge, Mass.), and “fitted” into the ligand-binding pockets (LBP) of ER-α (1 ERE) and ER-β (1 QKM) following manual superimposition using Deepview Swiss-PDB Viewer v.3.7 (GlaxoSmithKline and the Swiss Institute of Bioinformatics, see, e.g., on the internet at us.expasy.org/spdbv). Crystal structures were downloaded from the protein database (PDB). The final derivation depicting the lowest energy model predicts poor binding, yet preferential affinity for ER-β, as a result of steric restrictions corresponding primarily to amino acid Met 421, which resides within the LBP of ER-α. In addition, the minimized structure for APVE₂ corresponds well with previously published NMR derived structures previously published for this series of compounds (Sebag et al., J. Org. Chem., 65:7902-7912, 2000).

### Example 2-Cell Culture

DU145, LNCaP, PC3, and MCF-7 cells were obtained from the American Type Culture Collection (Manassas, Va.). DU145, PC3, and MCF-7 cells were maintained in Dulbecco's modified Eagle's medium/F-12 supplemented with 5% heat inactivated Fetal Calf Serum and Penicillin/Streptomycin (Invitrogen Life Technologies, Carlsbad, Calif.). LNCaP cells were maintained in RPMI 1640 medium supplemented in the same fashion. Cells were cultured in 75 cm² flasks (Falcon, BD Biosciences, Bedford, Mass.) at 37° C. and 5% CO₂. For the MTS assay (Example 3), five thousand cells were plated in each well of a 24 well plate (Falcon), were allowed to attach for a 48-hour period in the media described, and changed to a defined medium, (DMEM/F-12 or RPMI1640) supplemented with insulin (BD Biosciences). A 1000× solution of the compound of interest solubilized in DMSO (Sigma, Saint Louis, Mo.) was diluted in the defined media just prior to starting each experiment, and media was refreshed every 48 to 72 hours throughout the experiments.

### Example 3-MTS Assay

Cell viability assays were carried out with five logs of concentration between 0.1 nM and 1 μM for APVE₂, including controls in three prostate cancer cell lines (DU145, LNCaP, and PC3, and in a breast cancer cell line (MCF-7). Upon completing five days of treatment, the medium in each well was exchanged for 400 μL of an MTS solution (3-(4,5-dimethylthiazol-2-yl)-5-(3-carboxymethoxyphenyl)-2-(4-sulfophenyl)-2H-tetrazolium, inner salt; O'Toole SA, 2003, Cancer Detect Prev., 2003;27(1):47-54) spiked into media as per the manufacture's instructions (Promega, Madison, Wis.) and incubated for 2 hours at 37° C. before reading the absorbance on the μQuant UV/VIS plate reader, in conjunction with KCjunior Software (Bio-Tek Instruments, Winooski, Vt.) set at 490 nm. All experiments were carried out in quadruplicate, control wells were treated with the carrier DMSO. E₂, which is estradiol-17β (Steraloids®, Newport, R.I.) and resveratrol (Sigma) were used as controls. Resveratrol was chosen as it was found to cause cell death in the cell lines chosen in a very reproducible manner over the time period chosen. E₂ was used as no apparent cytotoxic or proliferative effects were noted in DU145 cells at the concentrations above 1 μM. EC₅₀ values were calculated with GraphPad Prism software (GraphPad Software Inc., San Diego, Calif.).

ER competition studies were carried out using the MTS assay as described in DU145 cells with E₂ as the competitor. The concentration of APVE₂ utilized was derived from the EC₅₀ concentration. Treatment with E₂ at concentrations of 1 μM was performed at 0 and 1 hour prior to the addition of 16 nM (EC₅₀) APVE₂. Each experiment was carried out with carrier, E₂, E₂ with APVE₂, and APVE₂ alone.

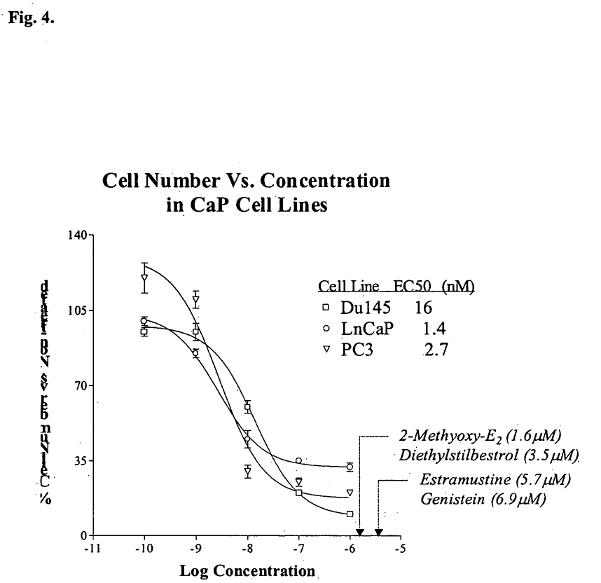

As shown in FIG. 4, APVE₂ caused a 92±0.9%, 81±0.8%, and 69±2.3% decrease in cell number, as determined by the MTS assay, in the DU145, PC3, and LNCaP cell lines, respectively, following 5 days of treatment at the initial screening dose of 1 μM. The negative control E₂ caused virtually no effect with an average of 0±3.4% difference in cell number over non-treated cells, while the positive control resveratrol induced an average of 65±5.3% decrease in cell number. These controls were added to each plate to control for any unforeseen variability. Incremental adjustments in APVE₂ concentrations from 10⁻¹⁰ M to 10⁻⁶ M resulted in a dose response with high statistical significance (r²=0.98 to 0.99) for each CaP cell line tested with EC₅₀ values of 16 nM, 1.4 nM, and 2.7 nM in the DU145, LNCaP, and PC3 cell lines, respectively (FIG. 4).

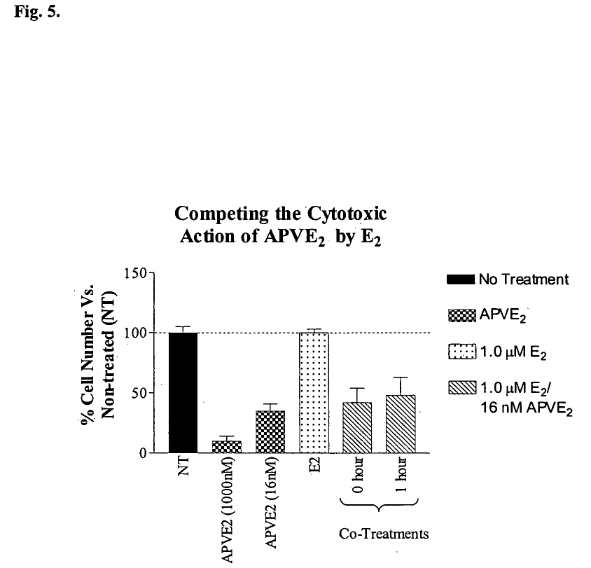

In addition, a 20% increase in cell proliferation occurred over non-treated controls at 1 nM in the PC3 cell line, but not in DU145 or LnCaP cell lines. Treatment of MCF-7 cells did not cause an increase in proliferation, but did induce cell death in a similar fashion to that of the other cell lines (data not shown). Since ER-β has been proposed to mediate the cytotoxic actions of anti-estrogens and SERMs, E₂ was used to determine if the cytotoxic action of APVE₂ could be competed by a classical estrogen (Kim et al., Cancer Res., 62:3649-3653, 2002; Lau et al., Cancer Res., 60:3175-3182, 2000). Our results indicate that the level of cytotoxicity induced by 16 nM APVE₂ is not reversed by co- or pre-treating DU145 cells with 1 μM E₂ at 0 or 1 hour respectively (FIG. 5).

### Example 4-Estrogen Receptor Relative Binding Affinity Assays

Binding affinities were determined by fluorescence polarization with the Victor 2 1420 FP analyzer set to FP @ 485 nm (Ex), 530 nm (Em) (Perkin Elmer), in conjunction with ER-α and ER-β kits (Pan Vera, Madison, Wis.) containing a fluorescent estrogen (E2S). All assays were carried out as per the manufacture's instructions with the following modifications. Briefly, 30 μl of competitor diluted in 10% DMF and the binding buffers supplied with the kit were combined with 30 μl of a solution containing 30 nM ER and 2 nM E2S in each well of the microplate (Corning 384 well black NBS # 3654). Each concentration was carried out in quadruplicate at 25° C. after equilibration for 2 hours, with E₂, 4-hydroxy-tamoxifen (4-OH-TAM), and DES used as controls. The effective concentration at 50% maximal activity (EC₅₀) was calculated for each compound and used to calculate the relative binding activities (RBAs).

The relative binding of APVE₂ was assessed by fluorescence polarimetry as described. The controls, DES and 4-OH-TAM, were found to exhibit RBAs that agree with previously published results for this assay, and APVE₂ was found to exhibit low RBAs to both of the receptors, ER-α and ER-β at 0.87% and 6.2%, respectively with a preference, for ER-β of ˜7 fold (Table 1, below).

TABLE 1
Relative Binding Activities to Two Estrogen Receptor Subtypes

| | ER-α | | ER-β | | |
|---|---|---|---|---|---|
| Compound | EC₅₀^{a} | % RBA^{b} | EC₅₀^{a} | % RBA^{b} | ER-α/ER-β |
| E₂^{c} | 3.19 | 100 | 15.5 | 100 | 1.0 |
| APVE₂^{c} | 365 | 0.87 | 250 | 6.2 | 0.14 |
| 4-OH-TAM^{c} | 13.2 | 24.2 | 59 | 26.3 | 0.92 |
| DES^{c} | 4.2 | 75.9 | 18.4 | 84.2 | 0.90 |

^{a}Effective concentrations at 50% half maximum are depicted in nM.
^{b}Relative binding is based on the ratio of EC₅₀ values as compared to E₂.
^{c}Average of four measurements at half-log concentrations from log −5.3 to −10.

### Example 5-Flow Cytometry/Apoptosis

DU145 cells were treated with APVE₂ at the EC₅₀ concentration of 16 nM according to the above mentioned protocol, with the exception that the experiment was terminated at 0, 1, and 2 days prior to the five day treatment. The cells were trypsinized for five minutes, scraped, washed in serum containing media, centrifuged at 300 rpm for 10 minutes, and fixed in ice-cold 70% ethanol for 24 hours at −20° C. The cells were again pelleted and resuspended in buffer containing nine parts 0.05M Na₂HPO₄ and one part 25 mM citric acid with 0.1% Triton X-100 (pH 7.8) for 2 hours. Cells were pelleted, resuspended in a buffer (10M PIPES, 0.1 N NaCl, 2 mM MgCl₂, and 0.1% Triton X-100) containing 20 μg PI and 50 μg/ml RNAse (pH 6.8), and incubated in the dark at room temperature for 30 minutes. Cell cycle distribution and DNA content analysis were assayed according to standard methods with a FACS sorter, FACScan™ flow cytometer (Becton Dickinson, Mountain View, Calif.), and analyzed by the Cell Cycle Multi-Cycle system (Phoenix Flow System, San Diego, Calif.). Approximately 15,000 single events were collected, and cell-cycle distribution was determined using Modfit LT version 2 software (Verity House, Inc., Topsham, Me.). All analyses were carried out in quadruplicate.

Apoptosis was assessed with the annexin-V-FITC apoptosis detection kit (Oncogene Research Products, Boston, Mass.) as per the manufacturer's instructions with E₂ and camptothecin (Calbiochem, San Diego, Calif.) as negative and positive controls respectively. Briefly, cells were lifted at the appropriate time points as described above and pelleted. Cells were then resuspended in ice cold binding buffer, and doubly stained with Annexin-V-fluorescein isothiocyanate and propidium iodide (PI) and analyzed within two hours on the FACScan™ flow cytometer (Becton Dickinson) and the Cell Cycle Multi-Cycle system (Phoenix Flow System).

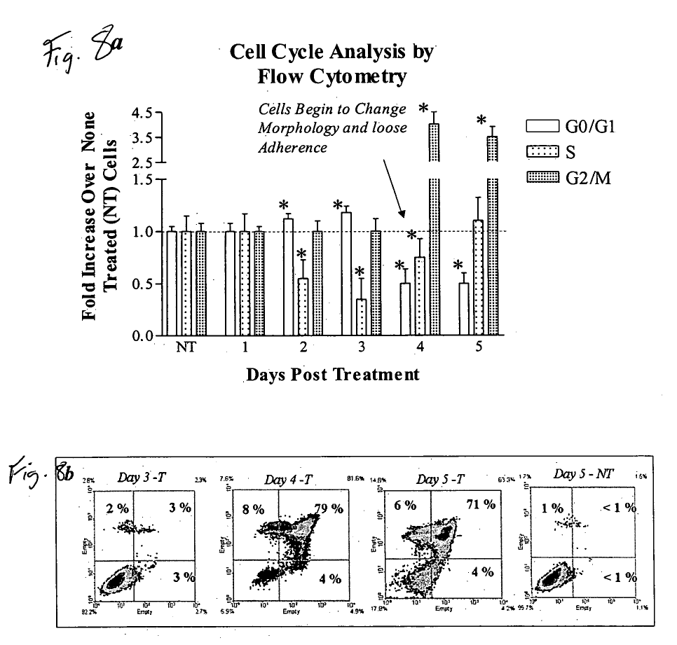

As shown in FIG. 8A, there was a significant increase in G1/G0 arrest by day 3 of 135% with a corresponding decrease in S phase to 32%, and no change in G2/M as compared to non-treated controls. By the 4^{th} day, cell morphology began to change with a rounding of cells and decreased adherence, corresponding with a sharp increase in G2/M arrest to 420%, a decrease in G1/G0 to 48%, and S phase to 73% over non-treated controls. Apoptosis was measured by co-staining live cells with PI and an Annexin V conjugated to a fluorescent tag. These results illustrate that the APVE₂ treated cells underwent G2/M arrest, which corresponded to a change in morphologic appearance on the 4^{th} and 5^{th} days of treatment correspond with approximately 76% of the cells staining heavily with both dyes, thus equating to late stage apoptosis (FIG. 8B).

### Example 6-Western Blot Analysis

DU145 and MCF-7 cells were cultured in 75 cm² flasks as described. At 75% confluence cells were lifted by trypsinization, washed in serum-containing media, followed by another wash in phosphate buffered saline (PBS), pelleted, and disrupted for protein extraction using the MPER extraction kit (Pierce, Rockford, Ill.) containing the protease inhibitor III cocktail kit (Calbiochem, San Diego, Calif.) as per the manufacture's instructions. Protein was quantified in quadruplicate using a BCA kit (Pierce, Rockford, Ill.) as per the manufacturer's instructions using a standard curve using bovine serum albumin (BSA) (Pierce, Rockford, Ill.) diluted to concentrations spanning the concentration of the samples. 25 μg of protein were added to each well of a 10-well 7.5% pre-poured acrylamide gel (BioRad, Hercules, Calif.); controls and treated samples were analyzed in triplicate. To determine specificity, 10 ng of each recombinant protein ER-αᵣ and ER-βᵣ (Pan Vera, Madison, Wis.) were blotted in the presence of each ER-specific antibody used. A kaleidoscope molecular weight standard (BioRad, Hercules, Calif.) was used to assess the relative size of the proteins. The gel was run at 80V until the running dye reached the bottom of the gel and electroblotted to a PVDF membrane (Whatman, Newton, Mass.) over a period of 1 hour at 250.mA.

The membrane was washed for 30 minutes in PBST (PBS +0.1% Tween 20) following the transfer, and placed on one of three rabbit primary antibodies, anti-ER-β (Biogenex, San Ramon, Calif.), anti-ER-α (Santa Cruz, Santa Cruz, Calif.), anti-β-actin (Sigma) for a period of 24 hours at 250:1, 2 hours at 500:1, and 1 hour at 1000:1 dilution, respectively at 4° C. in a solution of 0.1% BSA, and 5% milk in PBST. β-Actin was incubated with membranes pre-blotted and stripped as per standard protocol. The membranes were further washed in PBST for 30 minutes and incubated with a donkey anti-rabbit secondary antibody (Amersham, Piscataway, N.J.) in PBST for a period of 1 hour at 5000:1. Membranes were then rinsed and washed for four hours, and incubated in ECLPlus™ (Amersham, Piscataway, N.J.) for 10 minutes prior to imaging with a Storm™ 840 Scanner set to blue laser mode (Amersham, Piscataway, N.J.). ImageQuant™ (Amersham, Piscataway, N.J.) was utilized to process the image from the scanner and Kodak 1D software was then used to quantify the relative protein concentrations and molecular weight.

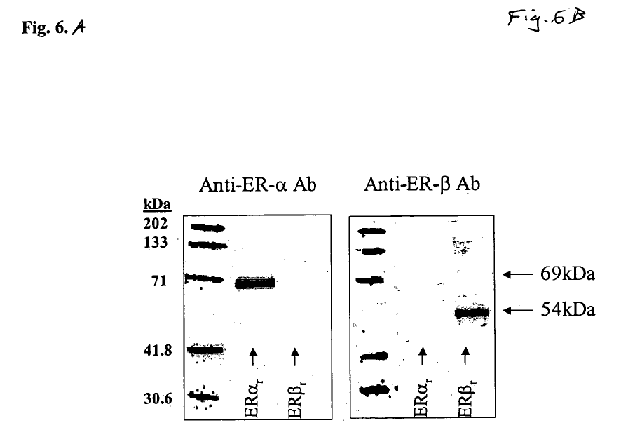

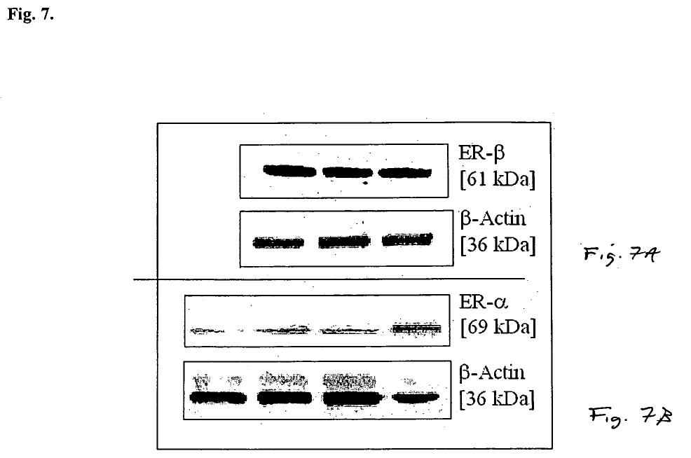

As shown in FIGS. 6A and 6B, no cross reactivity was observed for either of the two blots shown, and the calculated molecular weights agreed with the expected values of 69 kDa for ER-αᵣ and 54 kDa for ER-βᵣ. To determine the level of each ER at the protein level under the noted cell culture conditions, Western blotting was carried out. With 25 μg of total protein, the level of ER-α was barely detectible, while ER-β was expressed strongly (FIGS. 7A and 7B). β-Actin, used as a control for loading, was found to be equal in all samples tested, and 10 μg of cell extract from MCF-7 cells was used as a control for the ER-α blot.

### Example 7-Cytotoxicity Testing

Cytotoxicity Studies were done in DU-145 and MCF-7 Cells. The DU-145 cell line is an ER-β containing prostate cancer cell line that is devoid of ER-α and has been found to respond in a growth inhibitory/apoptotic fashion in the presence of estrogen analogs. We set out to test the efficacy of these compounds based on known affinities for ER-α previously reported. Cells were treated for five days with 1 mM of each compound in a defined media. Cell number was estimated by the MTS method. Similar studies were carried out in the MCF-7 cell line known to express both ER-α and -β, and display proliferative properties in the presence of classic estrogens. Various compounds were tested as indicated in Table 2 below.

TABLE 2

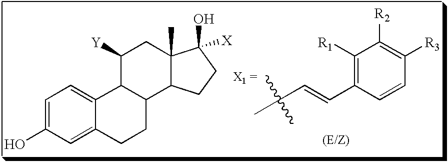

17α-(E/Z-(X-Phenyl)-Vinyl)-11β-(Y)-E₂

| Y | R₁ | R₂ | R₃ | E | Z |
|---|---|---|---|---|---|
| H, OCH₃ | H | H | H | ✓ | |
| H | H | H | CH₃ | ✓ | |
| H | H | CH₃ | H | ✓ | |
| H | H | H | F | ✓ | ✓ |
| H | F | H | H | | ✓ |
| H | H | H | CO₂CH₃ | ✓ | ✓ |
| H | CO₂CH₃ | H | H | ✓ | |
| H | H | H | CO₂H | ✓ | |
| H | CO₂H | H | H | ✓ | |
| H | H | H | CN | ✓ | |
| H | CH₂OH | H | H | ✓ | ✓ |
| H | H | H | OCH₃ | | ✓ |
| H | NO₂ | H | H | ✓ | |
| H | H | H | NO₂ | ✓ | |
| H | H | H | NH₃ | | ✓ |
| H, OCH₃ | H | H | CF₃ | ✓ | |
| H, OCH₃ | H | CF₃ | H | ✓ | |
| H, OCH₃ | CF₃ | H | H | ✓ | |

The results are shown in Table 3. In these tests, compound 7 is APVE₂. Other compounds that showed good results include 5, 9, 19, 23, and 25. Comparative tests with known estrogens and anti-estrogens are shown in Table 4.

TABLE 3

| | | | | | | MCF-7 | DU145 |
|---|---|---|---|---|---|---|---|
| ID # | Y | E/Z | R₁ | R₂ | R₃ | % Cell# | % Cell# |
| E₂ | H | — | — | — | — | 90 | 100 |
| 13* | H | — | — | — | — | 53 | 98 |
| 22* | H | E | — | — | — | 59 | 91 |
| 1 | H | E | H | H | H | 22 | 62 |
| 42 | CH₃O | E | H | H | H | 102 | 103 |
| 48 | H | E | H | H | H | 27 | 47 |
| 46 | H | E | H | H | NO₂ | 35 | 44 |
| 49 | H | E | NO₂ | H | H | 29 | 17 |
| 3 | H | E | H | H | CH₃ | 23 | 33 |
| 5 | H | E | H | CH₃ | H | 24 | 9 |
| 2 | H | Z | F | H | H | 40 | 80 |
| 6 | H | Z | H | H | F | 28 | 67 |
| 9 | H | E | H | H | F | 27 | 17 |
| 15 | H | Z | H | H | CO₂CH₃ | 32 | 92 |
| 4 | H | E | H | H | CO₂CH₃ | 28 | 100 |
| 12 | H | E | CO₂CH₃ | H | H | 30 | 92 |
| 8 | H | E | H | H | CO₂H | 120 | 99 |
| 24 | H | E | CO₂H | H | H | 61 | 103 |
| 7 | H | Z | H | H | NH₃ | 25 | 10 |
| 11 | H | Z | H | H | OCH₃ | 28 | 60 |
| 10 | H | E | H | H | CN | 23 | 92 |
| 16 | H | Z | CH₂OH | H | H | 55 | 105 |
| 20 | H | E | CH₂OH | H | H | 24 | 97 |
| 25 | H | E | H | H | CF₃ | 13 | 5 |
| 41 | CH₃O | E | H | H | CF₃ | 62 | 88 |
| 23 | H | E | H | CF₃ | H | 19 | 5 |
| 44 | CH₃O | E | H | CF₃ | H | 64 | 78 |
| 19 | H | E | CF₃ | H | H | 20 | 5 |
| 43 | CH₃O | E | CF₃ | H | H | 93 | 91 |

*(13) 17α-vinyl-E₂; (22) 17α-Br-vinyl-E₂

TABLE 4
Comparative Study w/Known Estrogens/Antiestrogens

| ID | | | | | MCF-7 | DU145 |
|---|---|---|---|---|---|---|
| # | Name | 7α | 11α | 17β | % Cell# | % Cell# |
| E₂ | Estradiol | H | H | H | 90 | 100 |
| 26 | RU 58668 | H | O(CH₂)ₓNH₂ | H | 37 | 73 |
| 27 | ICI 47, 699 | — | — | — | 21 | 12 |
| 28 | CI 680 | — | — | — | 48 | 15 |
| 29 | RU 1881 | H | H | CH₃ | 94 | 99 |
| 30 | TAM Citr | — | — | — | 33 | 47 |
| 31 | RU 39411 | O(CH₂)ₓNH₂ | H | H | 102 | 102 |
| 32 | ICI-182780 | (CH₂)ₓF | H | H | 62 | 94 |
| 33 | Z-TAM | — | — | — | 25 | 56 |
| 34 | 4-11, 100A | — | — | — | 32 | 29 |
| 35 | Danazol | H | H | CCN | 107 | 92 |
| 36 | (−) NGol | H | H | CCN | 104 | 103 |
| 37 | RU16117 | H | CH₃O | CCN | 103 | 101 |

### Example 8-In Vivo Toxicity Testing

NCR nude mice were used to test for general cytotoxicity effects in vivo of compounds 25 and 7 (Table 3). No apparent detrimental effects were observed to endpoint even at the higher doses over a 40-day period. Doses included 5 to 40 mg/kg/day bolus IM injection. Toxicity effects were gauged based on changes in animal weight and gross analysis of specific organs including liver, lung, prostate, brain, spleen, heart, and kidney, collected, and fixed in paraffin sections.

Xenotransplant studies are done using established prostate cancer cell lines (i.e. PC3, LNCaP, DU145) implanted using standard techniques in the same mouse model. In these studies, decreasing concentrations of test compounds are used to study the lowest-dose required for anti-tumor activity. Metabolism studies are carried out to follow the half-life of the test compounds of interest.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of inhibiting cell proliferation in a subject, the method comprising identifying a subject in which inhibition of cell proliferation is desirable; and administering to the subject a compound having the formula 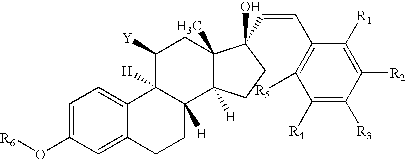
wherein R₆ is H or CH₃; R₁, R₂, R₄, and R₅ are, independently, H, NH₂, F, or CF₃; R₃ is H, NH₂, or F, and Y is H in an amount effective to inhibit cell proliferation in the subject.

2. The method of claim 1, wherein R₆ is hydrogen.

3. (canceled)

4. The method of claim 1, wherein the subject is a mammal.

5. The method of claim 1, wherein the subject is a human.

6. The method of claim 1, wherein the compound is administered intramuscularly.

7. The method of claim 1, wherein the compound is administered systemically.

8. The method of claim 1, wherein the compound is administered via an implant.

9. The method of claim 1, wherein the implant provides sustained release of the compound.

10. The method of claim 1, wherein the compound is administered intravenously.

11. The method of claim 1, wherein the cell proliferation is associated with cancer.

12. The method of claim 11, wherein the cancer is prostate or breast cancer.

13. The method of claim 1, wherein the amount of the compound administered is about 0.1 to 20 μg/kg body weight of the mammal per day.

14. The method of claim 1, wherein the compound is 17α-20Z-21-[(4-amino)phenyl]-19-norpregna-1,3,5(10),20-tetraene-3,17β-diol.

15. The method of claim 1, wherein
R₁, R₃, R₄, and R₅ are H, R₂ is CH₃;
R₁, R₂, R₄, and R₅ are H, R₃ is F;
R₂, R₃, R₄, and R₅ are H, R₁ is CF₃;
R₁, R₃, R₄, and R₅ are H, R₂ is CF₃; or
R₁, R₂, R₄, and R₅ are H, R₃ is CF₃.

16. A method of treating cancer, the method comprising administering to an individual having a cancer or at risk for having a cancer an amount of 17α-20Z-21-[(4-amino)phenyl]-19-norpregna-1,3,5(10),20-tetraene-3,17β-diol effective to inhibit proliferation of cells of the cancer.

17. The method of claim 16, wherein the cancer is prostate cancer.

18. The method of claim 16, wherein the cancer is breast cancer.

19. The method of claim 16, wherein the amount administered is in the range of from about 0.1 μg to about 40 mg/kg/day.

20-26. (canceled)

27. A method of treating prostate cancer by administering to an individual having prostate cancer or at risk for having prostate cancer a therapeutically effective amount of 17α-20E-21-[(4-fluoro)phenyl]-19-norpregna-1,3,5(10),20-tetraene-3,17-diol.
